(19) United States
(12) Patent Application Publication
(10) Pub. No.: US 2007/0173729 A1
(43) Pub. Date: Jul. 26, 2007

(54) Simple approach to precisely 02 consumption, and anesthetic absorption during low flow anesthesia

(76) Inventors: Joseph Fisher, Toronto (CA); David Prebes, Toronto (CA); Takekumi Azami, Toronto (CA); Alex Vesely, Toronto (CA); Eitan Prisman, Toronto (CA); Steve Coe, Toronto (CA); Ron Somogyi, Toronto (CA)

Correspondence Address: HERMAN & MILLMAN, 425 UNIVERSITY AVENUE, SUITE 300, TORONTO, ON, M5G 1T6, CA

(21) Appl. No.: 10/545,481

(22) PCT Filed: Feb. 18, 2004
(86) PCT No.: PCT/CA04/00219

§ 371 Date: Nov. 3, 2006

(30) Foreign Application Priority Data
Feb. 18, 2003 (CA) 2419103

(51) Int. Cl. A61B 5/08; A61M 16/00; A62B 7/00; F16K 31/02

(52) U.S. Cl. 600/532; 128/204.23; 128/204.22; 128/203.14

(57) ABSTRACT

A process for determining gas(x) consumption, wherein said gas(x) is selected from; a) an anesthetic such as but not limited to; i) N₂O; ii) sevoflurane; iii) isoflurane; iv) halothane; v) desflurame; or the like b) Oxygen (O₂).

### FIELD OF THE INVENTION

This invention relates to a method of intraoperative determination of O₂ consumption (\dot{V}O₂) and anesthetic absorption (VN₂O among others), during low flow anesthesia to provide information regarding the health of the patient and the dose of the gaseous and vapor anesthetic that the patient is absorbing. In addition to the monitoring function, this information would allow setting of fresh gas flows and anesthetic vaporizer concentration such that the circuit can be closed in order to provide maximal reduction in cost and air pollution.

The method provides an inexpensive and simple approach to calculating the flux of gases in the patient using information already available to the anesthesiologist The \dot{V}O₂ is an important physiologic indicator of tissue perfusion and an increase in \dot{V}O₂ may be an early indicator of malignant hyperthermia. The \dot{V}O₂ along with the calculation of the absorption/uptake of other gases would allow conversion to closed circuit anesthesia (CCA) and thereby save money and minimize pollution of the atmosphere.

### BACKGROUND OF THE INVENTION

A number of techniques exist which may be utilized to determine various values for oxygen flow or the like. Current methods of measuring gas fluxes breath-by-breath are not sufficiently accurate to close the circuit without additional adjustment of flows by trial and error. These prior techniques are set out below in the appropriate references. In the past many attempts have been made to measure VO₂ during anesthesia. The methods can be classified as:
1) Empirical formula based on body weight e.g.,
   a) The Brody equation (1) \dot{V}O₂=10*BW^{3/4} is a ‘static’ equation that cannot take into account changes in metabolic state.
2) Determination of oxygen loss (or replacement) in a closed system
   Severinghaus (2) measured the rate of N₂O and O₂ uptake during anesthesia. Patients breathed spontaneously via a closed breathing circuit (gas enters the circuit but none leaves). The flow of N₂O and O₂ into the circuit was continuously adjusted manually such that the total circuit volume and concentrations of O₂ and N₂O remain unchanged over time. If this is achieved, the flow of N₂O and O₂ will equal the rate of N₂O and O₂ uptake.
   Limitations: Unsuitable for clinical use.
      1. Method only works with closed circuit, which is seldom used clinically.
      2. Requires constant attention and adjustment of flows. This is incompatible with looking after other aspects of patient care during surgery.
      3. The circuit contains a device, a spirometer, that is not generally available in the operating room.
      4. Because the spirometer makes it impossible to mechanically ventilate patients, the method can be used only with spontaneously breathing patients.
      5. Method too cumbersome and imprecise to incorporate assessment of flux of other gases that are absorbed at smaller rates, such as anesthetic vapors.
3) Gas collection and measurement of O₂ concentrations:
   a) Breath-by-breath: measurement of O₂ concentration and expiratory flows at the mouth
      For this method, one of the commercially available metabolic carts can be attached to the patient's airway. Flow and gas concentrations are measured breath-by-breath. The device keeps a running tally of inspired and expired gas volumes.
   Limitations:
      1. Metabolic carts are expensive, costing US $30,000-$50,000.
   2. The methods they use to measure O₂ flux (VO₂) are fraught with potential errors. They must synchronize both flow and gas concentration signals. This requires the precise quantification of the time delay for the gas concentration curve and corrections for the effect of gas mixing in the sample line and time constant of the gas sensor. The error is greatest during inspiration when there are large and rapid variations in gas concentrations. We have not found any reports of metabolic carts used to measure \dot{V}O₂ during anesthesia with semi-closed circuit
      3. Metabolic carts do not measure fluxes in N₂O and anesthetic vapor.
      Our method measures flux of O₂ (VO₂), N₂O (VN₂O), and anesthetic vapor (VAA) with a semi-closed anesthesia circuit using the gas analyzer that is part of the available clinical set-up.
   b) Collecting gas from the airway pressure relief (APL) valve and analyzing it for volume and gas concentration. This will provide the volumes of gases leaving the circuit This can be subtracted from the volumes of these gases entering the circuit. This requires timed gas collection in containers and analysis for volume and concentration.
   Limitations
      i) The gas containers, volume measuring devices, and gas analyzers are not routinely available in the operating room.
      ii) The measurements are labor-intensive, distracting the anesthetist's attention from the patient.
4) Tracer gases
   Henegahan (3) describes a method whereby argon (for which the rate of absorption by, and elimination from, the patient is negligible) is added to the inspired gas of an anesthetic circuit at a constant rate. Gas exhausted from the ventilator during anesthesia is collected and directed to a mixing chamber. A constant flow of N₂ enters the mixing chamber. Gas concentrations sampled at the mouth and from the mixing chamber are analyzed by a mass spectrometer. Since the flow of inert gases is precisely known, the concentrations of the inert gases measured at the mouth and from the mixing chamber can be used to calculate total gas flow. This, together with concentrations of O₂ and N₂O, can be used to calculate the fluxes of these gases.

This method uses the principles of the indicator dilution method. It requires gases, flowmeters, and sensors not routinely available in the operating room, such as argon, N₂, precise flowmeters, a mass spectrometer, and a gas-mixing chamber.
5) \dot{V}O₂ from variations of the Foldes (1952) method:

$$Foldes formula \text{:} {F}_{I} {O}_{2} = \frac{{O}_{2} flow - {V O}_{2}}{FGflow - {V O}_{2}}$$

Where FIO₂ is the inspired fraction of O₂; O₂flow is the flow setting in ml/min (essentially equivalent to VO₂); VO₂ is the O₂ uptake as calculated from body weight and expressed in ml/min (essentially equivalent to VO₂); and FG flow is the fresh gas flow (FGF) setting in ml/min.
a) Biro (4) reasoned that since modern sensors can measure fractional airway concentrations, the Foldes equation can be used to solve for VO₂.

$$\dot{V} {O}_{2} = \frac{{O}_{2} flow - ( {F}_{I} {O}_{2} * FGflow )}{1 - {F}_{I} {O}_{2}}$$

where FGflow and O₂flow are obtained from the settings of the flowmeters.
Drawbacks of the approach:
   1. This approach requires knowing the FIO₂. FIO₂ varies throughout the breath and must be expressed as a flow-averaged value. This requires both flow sensors and rapid O₂ sensors at the mouth; it therefore has the same drawbacks as the metabolic cart type of measurements.
   2. Even if FIO₂ can be measured and timed volumes of O₂ calculated, its use in the equation given in the article is incorrect for calculating VO₂. Biro calculated VO₂ of 21 patients during elective middle ear surgery using his modification of the Foldes equation. His calculations were within an expected range of VO₂ as calculated from body weight but he did not compare his calculated VO₂values to those obtained with a proven method. Recently Leonard et al (5) compared the VO₂ as measured by the Biro method with a standard Fick method in 29 patients undergoing cardiac surgery. His conclusion was the Biro method is an “unreliable measure of systemic oxygen uptake” under anesthesia. We also compared the VO₂ as calculated by the Biro equation with our data from subjects in whom VO₂ was measured independently and found a poor correlation.
   b) Viale et al (6) calculated VO₂ from the formula
      VO₂=VE*(FIO₂*FEN₂/FIN₂−FEO₂)
   Where FIO₂ and FEO₂ are inspired and expired fractional concentrations of O₂, respectively; FIN₂ and FEN₂ are inspired and expired N₂ fractional concentrations, respectively.
   The method requires equipment not generally available in the operating room—a flow sensor at the mouth to calculate VE and a mass spectrometer to measure FEN₂ and FIN₂. Furthermore, it is then like the breath-by-breath analyzers in that means must be provided to integrate flows and gas concentrations in order to calculate flow-weighted inspired concentrations of O₂ and N₂.
   c) Bengston's method (7) uses a semi-closed circle circuit with constant fixed fresh gas flow consisting of 30% O₂ balance N₂O. VO₂ is calculated as
      \dot{V}O₂=\dot{V}fgO₂−0.45(\dot{V}fgN₂O)−(kg: 70.1000.t^{-0.5}))
      where \dot{V}fgO₂ is oxygen fresh gas flow; \dot{V}fgN₂O is the N₂O fresh gas flow and kg is the patient weight in kilograms. The method was validated by collecting the gas that exited the circuit and measuring the volumes and concentrations of component gases.
   Limitations of the method:
      i) N₂O absorption/uptake is not measured but calculated from patient's weight and duration of anesthesia.
      ii) The equation is valid only for a fixed gas concentration of 30% O₂, balance N₂.
      iii) The validation method requires collection of gas and measurement of its volume and gas composition.
6) Anesthetic absorption/uptake predicted from pharmacokinetic principles and characteristics of anesthetic agent
   a) The equation described by Lowe H J. The quantitative practice of anesthesia. Williams and Wilkins. Baltimore (1981), p 16
      \dot{V}AA=f*MAC*λ_{B/G}*Q*t^{-1/2}
      where VAA is the uptake of the anesthetic agent, f*MAC represents the fractional concentration of the anesthetic as a fraction of the minimal alveolar concentration required to prevent movement on incision,, λ_{B/G} is the blood-gas partition coefficient, Q is the cardiac output and t is the time.
      Limitations:
      i) In routine anesthesia, cardiac output (Q) is unknown.
      ii) The formula is based on empirical averaged values and does not necessarily reflect the conditions in a particular patient. For example, it does not take into account the saturation of the tissues, a factor that affects VAA.
   b) Lin C Y. (8) proposes the equation for uptake of anesthetic agent (\dot{V}AA)
      \dot{V}AA=\dot{V}A*FI*(1−FA/FI)
      Where \dot{V}AA is the uptake of the anesthetic agent; VA is the alveolar ventilation, FA is the alveolar concentration of anesthetic, and FI is the inspired concentration of anesthetic.
   Limitations:
   i) This formula cannot be used as VA is unknown with low flow anesthesia;
   ii) FI is complex and may vary throughout the breath so a volume-averaged value is required.
   iii) FI is not available with standard operating room analyzers.
7) Calculations directly from invasively-measured values
   a. Pestana (9) and Walsh (10) placed catheters into a peripheral artery and into the pulmonary artery. They used the oxygen content of blood sampled from these catheters and the cardiac output as measured by thermodilution from the pulmonary artery to calculate VO₂. They compared the results to those obtained by indirect calorimetry.
   Limitations
   i) The method uses monitors not routinely available in the operating room.
   ii) The placement of catheters in the vessels has associated morbidity and cost.

Summary Table

| | | | | | | Measures | | | | Can |
|---|---|---|---|---|---|---|---|---|---|---|
| | | | | | Uses | gas not | | | Based on | measure |
| | | Standard | | Requires | expired | available | | Wrong | prediction | absorp [symbol] ion |
| | | Anesthetic | Additional | additional | gas | on clinical | Uses | assumptions | from | of other |
| | | Circuit | Manipulation | measurements | collection | monitor | “F₁O₂” | or equation | pooled data | anesth [symbol] tic |
| Empirical | Brody | | | | | | | | Yes body | No |
| formula | | | | | | | | | weight | |
| | | | | | | | | | needed | |
| | Severinghaus | No. Uses | Yes. | Yes. | | | | | Yes | No |
| | | closed | Constant | Circuit | | | | | | |
| | | circuit | adjustment | volume | | | | | | |
| | | | of flow | | | | | | | |
| Metabolic | | | | Yes. Flow | Yes | | Yes | | | No |
| carts | | | | at the | | | | | | |
| | | | | mouth. | | | | | | |
| Timed gas | | No. | | Yes. | Yes | Yes, | | | | Yes |
| collection | | | | Volume. | | volumes | | | | |
| Tracer | Vaile | No. | | Yes. | Yes | Yes, | Yes | | Yes- | No |
| gases | | Inserted | | \dot{V}_{β} | | —N₂ | | | assumes | |
| | | nonre- | | | | | | | RQ | |
| | | breathing | | | | | | | | |
| | | valve to | | | | | | | | |
| | | separate | | | | | | | | |
| | | gases | | | | | | | | |
| | Heneghan | | | Yes. | Yes | Yes. | Yes | | | Possib [symbol] y |
| Foldes | Biro | | | | | | Yes | Yes | | No |
| | Bengson | No. | | | Yes. | | | Yes-only | Yes- | No. |
| | | | | | For | | | valid for | weight | |
| | | | | | validation | | | fixed | | |
| | | | | | | | | inspired | | |
| | | | | | | | | gas ratio | | |
| Pharmco- | Lowe | | | Yes. | | Yes | | Yes | Yes | Yes. |
| kinetic | | | | \dot{Q}-time | | | | | | |
| principles | | | | | | | | | | |
| | Lin | | | Yes. \dot{V}_{A} | | | Yes | Yes | | No |

[symbol]

Text missing or illegible when filed

### REFERENCE LIST

### Reference List

(1) Brody S. Bioenergetics and Growth. New York: Reinhold, 21945.
(2) Severinghaus J W. The rate of uptake of nitrous oxide in man. J Clin Invest 1954; 33:1183-1189.
(3) Heneghan C P, Gillbe C E, Branthwaite M A. Measurement of metabolic gas exchange during anaesthesia. A method using mass spectrometry. Br J Anaesth 1981; 53(1):73-76.
(4) Biro P. A formula to calculate oxygen uptake during low flow anesthesia based on FIO2 measurement. J Clin Monit Comput 1998; 14(2):141-144.
(5) Leonard I E, Weitkamp B, Jones K, Aittomaki J, Myles P S. Measurement of systemic oxygen uptake during low-flow anaesthesia with a standard technique vs. a novel method. Anaesthesia 2002; 57(7):654-658.
(6) Viale J P, Annat G J, Tissot S M, Hoen J P, Butin E M, Bertrand O J et al. Mass spectrometric measurements of oxygen uptake during epidural analgesia combined with general anesthesia. Anesth Analg 1990; 70(6):589-593.
(7) Bengtson J P, Bengtsson A, Stenqvist O. Predictable nitrous oxide uptake enables simple oxygen uptake monitoring during low flow anaesthesia. Anaesthesia 1994; 49(1):29-31.
(8) Lin C Y. [Simple, practical closed-circuit anesthesia]. Masui 1997; 46(4):498-505.
(9) Pestana D, Garcia-de-Lorenzo A. Calculated versus measured oxygen consumption during aortic surgery: reliability of the Fick method. Anesth Analg 1994; 78(2):253-256.
(10) Walsh T S, Hopton P, Lee A. A comparison between the Fick method and indirect calorimetry for determining oxygen consumption in patients with fulminant hepatic failure. Crit Care Med 1998; 26(7):1200-1207.
11. Baum J A and Aitkenhead R A. Low-flow anaesthesia. Anaesthesia 50 (supplement): 37-44, 1995

### OBJECTS OF THE INVENTION

It is therefore a primary object of this invention to provide an improved method of intraoperative determination of O₂ consumption (\dot{V}O₂) and anesthetic absorption (VN₂O, among others), during low flow anesthesia to provide information regarding the health of the patient and the dose of the gaseous and vapor anesthetic that the patient is absorbing.

It is yet a further object of this invention to provide, based on determination of O₂ consumption (\dot{V}O₂) and anesthetic absorption (VN₂O, among others), the setting of fresh gas flows and anesthetic vaporizer concentration such that the circuit can be substantially closed in order to provide maximal reduction in cost and air pollution.

Further and other objects of the invention will become apparent to those skilled in the art when considering the following summary of the invention and the more detailed description of the preferred embodiments illustrated herein.

### BRIEF DESCRIPTION OF THE FIGURES

FIG. 1 is a Bland-Altman plot showing the precision of the calculated oxygen consumption compared to the actual “oxygen consumption” simulation in a model, labeled as “virtual \dot{V}O₂”.

### SUMMARY OF THE INVENTION

According to a primary aspect of the invention, there is provided a method to precisely calculate the flux of O₂ (VO₂) and anesthetic gases such as N₂O (VN₂O) during steady state low flow anesthesia with a semi-closed or dosed circuit such as a circle anesthetic circuit or the like. For our calculations, we require only the gas flow settings and the outputs of a tidal gas analyzer. We will consider a patient breathing via a circle circuit with fresh gas consisting of O₂ and/or air, with or without N₂O, entering the circuit at a rate substantially less than the minute ventilation (\dot{V}E). We will refer to the total fresh gas flow (FGF) as “source gas flow” (SGF). Our perspective throughout will be that the circuit is an extension of the patient and that under steady state conditions, the mass balance of the flux of gases with respect to the circuit is the same as the flux of gases in the patient.

We present an approach that increases the precision of gas flux calculations for determining gas pharmacokinetics during low flow anesthesia, one application of which is to institute CCA. According to one aspect of the invention there is provided a process for determining gas(x) consumption, wherein said gas(x) is selected from;
a) an anesthetic such as but not limited to;
   i) N₂O;
   ii) sevoflurane;
   iii) isoflurane;
   iv) halothane;
   v) desflurame; or the like
b) Oxygen (O₂);

for example, in a semi-closed or closed circuit, or the like comprising the following relationships;

wherein said relationships are selected from the groups covering the following circumstances;

Model 1

As an initial simplifying assumption, we consider that the CO₂ absorber is out of the circuit and the respiratory quotient (RQ) is 1.

We can make a number of statements with regard to Model 1:
1) The flow of gas entering the circuit is SGF and the flow of gas leaving the circuit is equal to SGF.
2) The gas leaving the circuit is predominantly alveolar gas. This is substantially true as the first part of the exhaled gas that contains anatomical dead-space gas would tend to bypass the pressure relief valve and enter the reservoir bag. When the reservoir bag is full, the pressure in the circuit will rise, thereby opening the pressure relief valve, allowing the later-expired gas from the alveoli to exit the circuit.
3) The volume of any gas ‘x’ entering the circuit can be calculated by multiplying SGF times the fractional concentration of gas x in SGF (FSX). The volume of gas x leaving the circuit is SGF times the fractional concentration of x in end tidal gas (FETX). The net volume of gas x absorbed by, or eliminated from, the patient is SGF (FSX−FETX). For example, \dot{V}O₂=SGF (FSO₂−FETO₂) where SGF and FSO₂ can be read from the flow meter and FETO₂ is read from the gas monitor. Similar calculations can be used to calculate \dot{V}CO₂ and the flux of inhaled anesthetic agents.
   Model 2

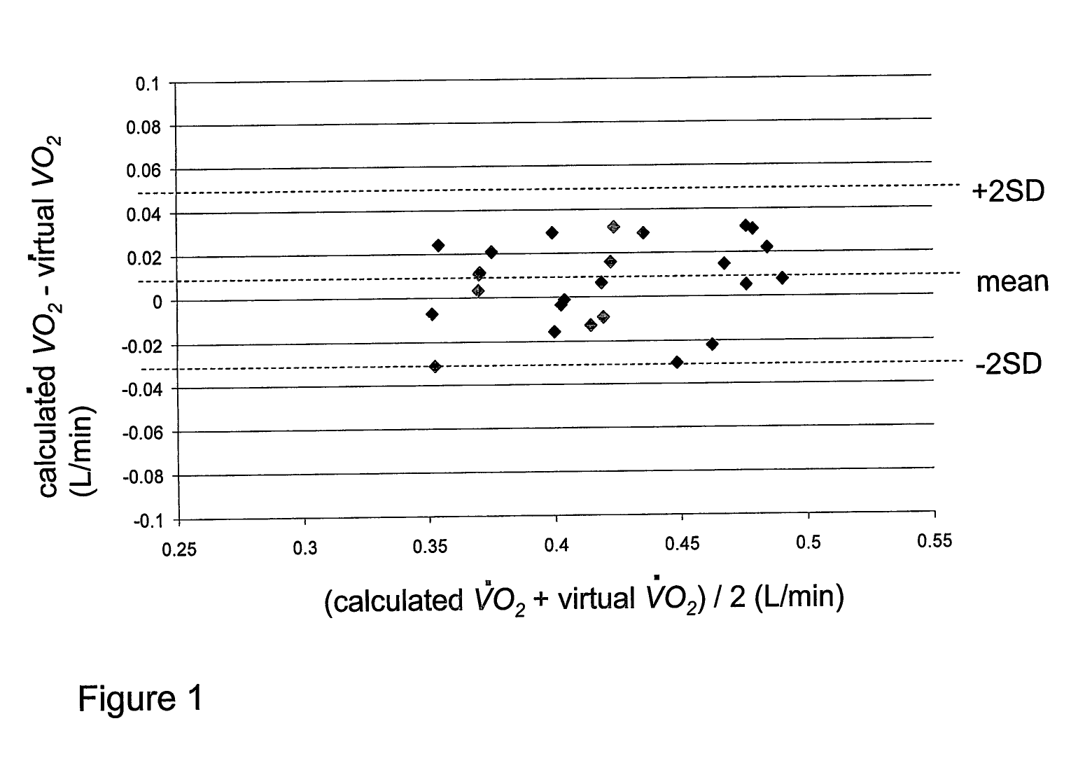

We will now consider a circle circuit with a CO₂ absorber in the circuit. As an initial simplifying assumption, we will assume that all of the expired gas passes through the CO₂ absorber and RQ is 1 (see FIG. 1b).

With this model, all of the CO₂ produced by the patient is absorbed, so the total flow of gas out of the circuit (Tfout; equivalent to the expiratory flow, VE) is no longer equal to SGF but equal to SGF minus \dot{V}O₂.
TFout=SGF−\dot{V}O₂ (1)

\dot{V}O₂ is calculated as the flow of O₂ into the circuit (O₂in; equivalent in standard terminology to VO₂in) minus the flow of O₂ out of the circuit (O₂out; equivalent in standard terminology to VO₂out).
\dot{V}O₂=O₂in−O₂out (2)
Since,
O₂out=TFout*FETO₂ (3)
then simply by substituting (3) for O₂out in (2) we can calculate \dot{V}O₂ from the gas settings and the O₂ gas monitor reading:
\dot{V}O₂═SGF*(FSO₂−FETO₂)/(1−FETO₂) (4)
Model 3

We will again consider the case of anesthesia provided via a circle circuit with a CO₂ absorber in the circuit. In this model we will take into account that some expired gas escapes through the pressure relief valve (FIG. 2) and some passes through the CO₂ absorber. The RQ is still assumed to be 1. We will ignore for the moment the effect of anatomical dead-space and assume all gas entering the patient contributes to gas exchange. We will assume that during inhalation the patient receives all of the SGF and the balance of the inhaled gas in the alveoli comes from the expired gas reservoir after being drawn through the CO₂ absorber.

An additional simplifying assumption is that the volume of gas passing through the CO₂ absorber is the difference between \dot{V}E and the SGF (i.e., \dot{V}_{E}−SGF)¹. The proportion of previous exhaled gas passing through the CO₂ absorber that is distributed to the alveoli is 1−SGF/\dot{V}E². We will call this latter proportion ‘a’.
¹ In fact, it is the \dot{V}E−SGF+\dot{V}CO₂ abs. The difference between this value and our assumption is so small that we will ignore it for now

² Why this is not strictly true is described in the discussion about Model 4; absorption of CO₂ increases the concentrations of other gases.

a=1−SGF/\dot{V}E (5)

As before, we know the flows and concentrations of gases entering the circuit. To calculate the flow of individual gases leaving the circuit we need to know the total flow of gas out of the circuit. In this model we account for the volume of CO₂ absorbed by the CO₂ absorber. We still assume RQ=1. The flow out of the circuit is equal to the SGF minus the \dot{V}O₂ plus the \dot{V}CO₂, minus the volume of CO₂ in the gas that is drawn through the CO₂ absorber (\dot{V}CO₂abs):
Tfout=SGF−\dot{V}O₂+\dot{V}CO₂−\dot{V}CO₂abs (6)

Recall that \dot{V}CO₂abs=a \dot{V}CO₂
TFout=SGF−\dot{V}O₂+\dot{V}CO₂−a \dot{V}CO₂
\dot{V}O₂=O₂in−O₂ out
\dot{V}O₂=O₂ in−(SGF−\dot{V}O₂+\dot{V}CO₂−a \dot{V}CO₂)FETO₂

As the RQ is assumed to be 1, we can substitute \dot{V}O₂ for \dot{V}CO₂ and VE for VI and solve for \dot{V}O₂:

$$\dot{V} {O}_{2} = \frac{{O}_{2} in - SGF \times {F}_{ET} {O}_{2}}{1 - ( 1 - \frac{SGF}{\dot{V} E} ) {F}_{ET} {O}_{2}} \qquad ( 7 )$$

In addition, we amend the equations to account for the actual RQ, if known. When we assumed that RQ=1, we were able to simply substitute \dot{V}O₂ for \dot{V}CO₂. To correct for RQ other than 1, we now use \dot{V}CO₂=RQ*\dot{V}O₂ and \dot{V}CO₂ abs is therefore equal to a*RQ*VO₂. Therefore
TFout=SGF−\dot{V}O₂+\dot{V}CO₂−\dot{V}CO₂abs (6)
becomes
TFout=SGF−\dot{V}O₂+RQ \dot{V}O₂−a*RQ*\dot{V}O₂ (8)

In the case of a second gas being absorbed, such as N₂O or anesthetic vapor, a similar equation can be written in which the total flow out (TFout) also includes a term correcting for the flux of N₂O (\dot{V}N₂O) and/or anesthetic agent (VAA).

Therefore for Model 3 with calculations of \dot{V}N₂O absorption (\dot{V}N₂O) and RQ=1

In model 3, adding terms for the calculation of \dot{V}N₂O to equation (6) while assuming RQ=1,
TFout=SGF−\dot{V}O₂−\dot{V}N₂O+\dot{V}CO₂−\dot{V}CO₂abs (AA1)
In order to determine the \dot{V}N₂O, a second mass balance equation about the circuit with respect to N₂O is required. For \dot{V}CO₂abs=a*\dot{V}CO₂ and a=1−SGF/\dot{V}E
\dot{V}N₂O=N₂O in−(SGF−\dot{V}O₂−\dot{V}N₂O+\dot{V}CO₂−a*\dot{V}CO₂)*FETN₂O (AA2)

As RQ is still assumed to equal 1, \dot{V}O₂=\dot{V}CO₂

$$\begin{matrix}\dot{V} {N}_{2} O = {N}_{2} O in - ( \begin{matrix}SGF - \dot{V} {O}_{2} - \dot{V} {N}_{2} O + \\ {\dot{V}}^{} {O}_{ 2} - a \dot{V} {O}_{ 2}\end{matrix} ) * {F}_{ET} {N}_{ 2} O \\ = {N}_{2} O i n - ( SGF - a \dot{V} {O}_{2} - \dot{V} {N}_{2} O ) * {F}_{ET} {N}_{2} O\end{matrix} \qquad ( AA 3 )$$

Therefore when taking \dot{V}N₂O into account, \dot{V}O₂ can be recalculated as

$$\begin{matrix}\dot{V} {O}_{2} = {O}_{2} i n - ( \begin{matrix}SGF - \dot{V} {O}_{2} - \dot{V} {N}_{2} O + \\ \dot{V} {CO}_{ 2} - a * \dot{V} {CO}_{ 2}\end{matrix} ) * {F}_{ET} {O}_{2} \\ = {O}_{2} i n - ( \begin{matrix}SGF - \dot{V} {O}_{2} - \dot{V} {N}_{2} O + \\ \dot{V} {O}_{ 2} - a \dot{V} {O}_{ 2}\end{matrix} ) * {F}_{ET} {O}_{2} \\ = {O}_{2} i n - ( SGF - a \dot{V} {O}_{2} - \dot{V} {N}_{2} O ) * {F}_{ET} {O}_{2}\end{matrix} \qquad ( AA 4 )$$

Basically, we have two equations, (AA3) and (AA4) with two unknowns, \dot{V}O₂ and \dot{V}N₂O.
Solving equation (AA3) for \dot{V}N₂O,

$$\dot{V} {N}_{2} O = \frac{{N}_{2} O i n - ( SGF - a \dot{V} {O}_{2} ) * {F}_{ET} {N}_{2} O}{1 - {F}_{ET} {N}_{2} O} \qquad ( AA 5 )$$

Substituting (AA5) into equation (AA4) and solving for \dot{V}O₂,

$$\dot{V} {O}_{2} = \frac{( 1 - {F}_{ET} {N}_{2} O ) * {O}_{2} i n - ( SGF - {N}_{2} O i n ) * {F}_{ET} {O}_{2}}{1 - ( 1 - \frac{SGF}{\dot{V} E} ) * {F}_{ET} {O}_{2} - {F}_{ET} {N}_{2} O} \qquad ( AA 6 )$$

And calculating \dot{V}N₂O taking into account \dot{V}O₂, CO₂ absorption and RQ=1:

$$\dot{V} {N}_{2} O = \frac{\begin{matrix}( 1 - ( 1 - \frac{SGF}{\dot{V} E} ) * {F}_{ET} {O}_{2} ) * {N}_{2} O i n - \\ ( SGF - {O}_{2} i n ) * {F}_{ET} {N}_{2} O\end{matrix}}{1 - ( 1 - \frac{SGF}{\dot{V} E} ) * {F}_{ET} {O}_{2} - {F}_{ET} {N}_{2} O} \qquad ( AA 7 )$$

$$\begin{matrix}\dot{V} {O}_{2} = \frac{\begin{matrix}( 1 - {F}_{ET} {N}_{2} O - {F}_{ET} AA ) * {O}_{2} i n - \\ ( SGF - {N}_{2} O i n - AA i n ) * {F}_{ET} {O}_{2}\end{matrix}}{1 - a * {F}_{ET} {O}_{2} - {F}_{ET} {N}_{2} O - {F}_{ET} AA} & ( AA 8 ) \\ \dot{V} {N}_{2} O = \frac{\begin{matrix}( 1 - a * {F}_{ET} {O}_{2} - {F}_{ET} AA ) * {N}_{2} O i n - \\ ( SGF - a * {O}_{2} i n - AA i n ) * {F}_{ET} {N}_{2} O\end{matrix}}{1 - a * {F}_{ET} {O}_{2} - {F}_{ET} {N}_{2} O - {F}_{ET} AA} & ( AA 9 ) \\ \dot{V} AA = \frac{\begin{matrix}( 1 - a * {F}_{ET} {O}_{2} - {F}_{ET} {N}_{2} O ) * AA i n - \\ ( SGF - a * {O}_{2} i n - {N}_{2} O i n ) * {F}_{ET} AA\end{matrix}}{1 - a * {F}_{ET} {O}_{2} - {F}_{ET} {N}_{2} O - {F}_{ET} AA} \\ where a = 1 - \frac{SGF}{\dot{V} E} & ( AA 10 )\end{matrix}$$

Model 3 with N2O, RQ

Taking into account the actual RQ while calculating \dot{V}N₂O, equation 9 becomes,
TFout=SGF−\dot{V}O₂−\dot{V}N₂O+RQ \dot{V}O₂−a*RQ* \dot{V}O₂ (AA11)

Therefore equation (AA2) becomes,
\dot{V}N₂O=N₂O in −(SGF−\dot{V}O₂−\dot{V}N₂O+RQ \dot{V}O₂−a*RQ*\dot{V}O₂)*FETN₂O (AA12)

And equation (AA4) becomes,
\dot{V}O₂=O₂in −(SGF−\dot{V}O₂−\dot{V}N₂O+RQ \dot{V}O₂−a*RQ*\dot{V}O₂)*FETO₂ (AA13)

Now, we have two equations, (AA12) and (AA13) with two unknowns, \dot{V}O₂ and \dot{V}N₂O.

Solving equation (AA12) and (AA13) for \dot{V}O₂ and \dot{V}N₂O,

$$\begin{matrix}\dot{V} {O}_{2} = \frac{( 1 - {F}_{ET} {N}_{2} O ) * {O}_{2} i n - ( SGF - {N}_{2} O i n ) * {F}_{ET} {O}_{2}}{1 - b * {F}_{ET} {O}_{2} - {F}_{ET} {N}_{2} O} & ( AA 14 ) \\ \dot{V} {N}_{2} O = \frac{\begin{matrix}( 1 - b * {F}_{ET} {O}_{2} ) * {N}_{2} O i n - \\ ( SGF - {O}_{2} i n ) * {F}_{ET} {N}_{2} O\end{matrix}}{1 - b * {F}_{ET} {O}_{2} - {F}_{ET} {N}_{2} O} & ( AA 15 )\end{matrix}$$

where b is the fraction of the CO₂ production (VCO₂) passing through the CO₂ absorber. “b” is analogous to “a” and is formulated to account for the actual RQ.

$$b = 1 - RQ ( 1 - ( 1 - \frac{SGF}{\dot{V} E} ) ) = 1 - RQ * \frac{SGF}{\dot{V} E}$$

Model 3 with N₂O and Anesthetic Agent, RQ

Similarly, the flux of gases can be calculated taking into account the actual RQ.

$$\begin{matrix}\dot{V} {O}_{2} = \frac{\begin{matrix}( 1 - {F}_{ET} {N}_{2} O - {F}_{ET} AA ) * {O}_{2} i n - \\ ( SGF - {N}_{2} O i n - AA i n ) * {F}_{ET} {O}_{2}\end{matrix}}{1 - b * {F}_{ET} {O}_{2} - {F}_{ET} {N}_{2} O - {F}_{ET} AA} & ( AA16 ) \\ \dot{V} {N}_{2} O = \frac{\begin{matrix}( 1 - b * {F}_{ET} {O}_{2} - {F}_{ET} AA ) * {N}_{2} O i n - \\ ( SGF - b * {O}_{2} i n - AA i n ) * {F}_{ET} {N}_{2} O\end{matrix}}{1 - b * {F}_{ET} {O}_{2} - {F}_{ET} {N}_{2} O - {F}_{ET} AA} \\ \dot{V} AA = \frac{\begin{matrix}( 1 - b * {F}_{ET} {O}_{2} - {F}_{ET} {N}_{2} O ) * AA i n - \\ ( SGF - b * {O}_{2} i n - {N}_{2} O i n ) * {F}_{ET} AA\end{matrix}}{1 - b * {F}_{ET} {O}_{3} - {F}_{ET} {N}_{2} O - {F}_{ET} AA} & ( AA17 )\end{matrix}$$

Model 4

The one remaining simplifying assumption is that we have ignored the effects of the anatomical dead-space.

We know the portion of the inspired gas that passes through the CO₂ absorber as \dot{V}E-SGF. However, the net amount of CO₂ absorbed by the CO₂ absorber will be equal to that contained in the portion of the \dot{V}E-SGF that originated from the alveoli on a previous breath. The gas from the alveoli has a FCO₂ equal to FETCO₂. Therefore, the proportion of inhaled gas drawn through the CO₂ absorber we had previously designated as ‘a’ is actually equal to 1−SGF/\dot{V}A. To avoid confusion in subsequent derivations we will designate 1−SGF/\dot{V}A as a′.

We now amend equation (7) removing simplifying assumptions about RQ and using a′ as the proportion of gas passing the CO₂ absorber.

Now,
\dot{V}O₂abs=a*\dot{V}O₂=(1−SGF/\dot{V}A)*\dot{V}O₂ (9)

From equation (8),

$$\begin{matrix}TFout = SGF - \dot{V} {O}_{2} + \dot{V} {CO}_{2} - \dot{V} {CO}_{2} abs \\ = SGF - \dot{V} {O}_{2} + ( 1 - {a}^{′} ) * \dot{V} {CO}_{2} \\ = SGF - \dot{V} {O}_{2} + ( 1 - ( 1 - SGF / {\dot{V}}_{A} ) ) * \dot{V} {CO}_{2} \\ = SGF - \dot{V} {O}_{2} + ( SGF / \dot{V} A ) * \dot{V} {CO}_{2} \\ = SGF - \dot{V} {O}_{2} + SGF * ( \dot{V} {CO}_{2} / {\dot{V}}_{A} )\end{matrix} \qquad ( 10 )$$

As the standard definition of FETCO₂ is \dot{V}CO₂/\dot{V}A, we substitute \dot{V}CO₂/\dot{V}A for FETCO₂ in (10)

$$TFout = SGF - \dot{V} {O}_{2} + SGF * FET {CO}_{2}$$

$$\begin{matrix}\dot{V} {O}_{2} = {O}_{2} i n - TFout * FET {O}_{2} \\ = {O}_{2} i n - ( SGF - \dot{V} {O}_{2} + SGF * FET {CO}_{2} ) * FET {O}_{2}\end{matrix}$$

After isolating \dot{V}O2

$$V O 2 = \frac{O 2 i n - ( SGF + SGF * FET CO 2 ) * FET {O}_{2}}{1 - FET {O}_{2}} \qquad ( 11 )$$

Model 4 Amended for VN2O

Amending equation (11) for \dot{V}N₂O
TFout=SGF−\dot{V}O₂−\dot{V}N₂O+\dot{V}CO₂−\dot{V}CO₂abs

In order to determine the \dot{V}N₂O, a second mass balance about N2O is required: where \dot{V}CO₂abs=a′*\dot{V}CO₂ and a′=1−SGF/\dot{V}A

$$\begin{matrix}\dot{V} {N}_{2} O = {N}_{2} O i n - ( \begin{matrix}SGF - \dot{V} {O}_{2} - \dot{V} {N}_{2} O + \\ \dot{V} {CO}_{2} - {a}^{′} * \dot{V} {CO}_{2}\end{matrix} ) * {F}_{ET} {N}_{2} O \\ = {N}_{2} O i n - ( \begin{matrix}SGF - \dot{V} {O}_{2} - \dot{V} {N}_{2} O + \\ ( 1 - {a}^{′} ) * \dot{V} {CO}_{2}\end{matrix} ) * {F}_{ET} {N}_{2} O \\ = {N}_{2} O i n - ( \begin{matrix}SGF - \dot{V} {O}_{2} - \dot{V} {N}_{2} O + \\ ( 1 - ( 1 - SGF / {\dot{V}}_{A} ) * \dot{V} {CO}_{2} )\end{matrix} ) * {F}_{ET} {N}_{2} O \\ = {N}_{2} O i n - ( \begin{matrix}SGF - \dot{V} {O}_{2} - \dot{V} {N}_{2} O + \\ SGF / {\dot{V}}_{A} * \dot{V} {CO}_{2}\end{matrix} ) * {F}_{ET} {N}_{2} O \\ = {N}_{2} O i n - ( \begin{matrix}SGF - \dot{V} {O}_{2} - \dot{V} {N}_{2} O + \\ SGF * {F}_{ET} CO 2\end{matrix} ) * {F}_{ET} {N}_{2} O\end{matrix} \qquad ( 28 )$$

In the same way,

$$\begin{matrix}\dot{V} {O}_{2} = {O}_{2} i n - ( \begin{matrix}SGF - \dot{V} {O}_{2} - \dot{V} {N}_{2} O + \\ \dot{V} {CO}_{2} - {a}^{′} * \dot{V} {CO}_{2}\end{matrix} ) * {F}_{ET} {O}_{2} \\ = {O}_{2} i n - ( \begin{matrix}SGF - \dot{V} {O}_{2} - \dot{V} {N}_{2} O + \\ SGF * {F}_{ET} CO 2\end{matrix} ) * {F}_{ET} {O}_{2}\end{matrix} \qquad ( 29 )$$

Now, we have two equations, (28) and (29) with two unknowns, \dot{V}O₂ and \dot{V}N₂O. Solving equation (28) and (29) for \dot{V}O₂ and \dot{V}N₂O,

$$\begin{matrix}\dot{V} {O}_{2} = \frac{\begin{matrix}{O}_{2} i n * ( 1 - {F}_{ET} {N}_{2} O ) - \\ ( SGF * ( 1 + {F}_{ET} {CO}_{2} ) - {N}_{2} O i n ) * \\ {F}_{ET} {O}_{2}\end{matrix}}{1 - FET {N}_{2} O - FET {O}_{2}} & ( 30 ) \\ \dot{V} {N}_{2} O = \frac{\begin{matrix}{N}_{2} O i n * ( 1 - {F}_{ET} {O}_{2} ) - \\ ( SGF * ( 1 + {F}_{ET} {CO}_{2} ) - O i n ) * \\ {F}_{ET} {N}_{2} O\end{matrix}}{1 - {F}_{ET} {N}_{2} O - {F}_{ET} {O}_{2}} & ( 31 )\end{matrix}$$

Note that RQ and \dot{V}A are not required to calculate flux. We present the equations where equation 11 is further amended to take into account \dot{V}N₂O and \dot{V}AA.

$$\dot{V} O 2 = \frac{\begin{matrix}O 2 {in}^{*} ( 1 - {FET}_{2} NO - {FETAAFET}_{2} {NO}^{*} {FETAA}_{2} ) \\ ( {SGF}^{*} ( 1 + {FET}_{2} CO ) - {N}_{2} Oin - \\ {AAin}_{2} {FET}_{2} {NO}^{*} {FETAA}^{*} ( 1 - \\ {{N}_{2} Oin - AAin )}^{*} ) {FET}_{2} O\end{matrix}}{\begin{matrix}{( 1 - {FET}_{2} NO )}^{*} ( 1 - {FETAA}_{2} ) \\ ( 1 - {FET}_{2} {NO}^{*} {FETAA}^{*} ) {FET}_{2} O\end{matrix}} \\ VNO = \frac{\begin{matrix}{N}_{2} {Oin}^{*} ( 1 - {FET}_{2} O - FETAA - {FET}_{2} {O}^{*} FETAA ) - \\ ( {SGP}^{*} ( 1 + {FET}_{2} CO ) - {O}_{2} in - AAin - \\ {FET}_{2} {O}^{*} {{FETAA}^{*} ( 1 - {O}_{2} in - AAin )}^{*} ) {FET}_{2} N \text{?}\end{matrix}}{\begin{matrix}{( 1 - {FET}_{2} O )}^{*} ( 1 - FETAA ) - \\ {( 1 - {FET}_{2} {O}^{*} FETAA )}^{*} {FET}_{2} NO\end{matrix}} \\ \text{?} \text{indicates text missing or illegible when filed} \qquad ( 11 )$$

Model 4 with N2O and Anesthetic Agent

Similarly, the flux of additional anesthetic agents can be calculated by adding more

$$\dot{V} O 2 = \frac{\begin{matrix}O 2 {in}^{*} ( 1 - {FET}_{2} NO - {FETAAFET}_{2} {NO}^{*} {FETAA}_{2} ) \\ ( {SGF}^{*} ( 1 + {FET}_{2} CO ) - {N}_{2} Oin - \\ {AAin}_{2} {FET}_{2} {NO}^{*} {FETAA}^{*} ( 1 - \\ {{N}_{2} Oin - AAin )}^{*} ) {FET}_{2} O\end{matrix}}{\begin{matrix}{( 1 - {FET}_{2} NO )}^{*} ( 1 - {FETAA}_{2} ) \\ ( 1 - {FET}_{2} {NO}^{*} {FETAA}^{*} ) {FET}_{2} O\end{matrix}} \\ VNO = \frac{\begin{matrix}{N}_{2} {Oin}^{*} ( 1 - {FET}_{2} O - FETAA - {FET}_{2} {O}^{*} FETAA ) - \\ ( {SGP}^{*} ( 1 + {FET}_{2} CO ) - {O}_{2} in - AAin - \\ {FET}_{2} {O}^{*} {{FETAA}^{*} ( 1 - {O}_{2} in - AAin )}^{*} ) {FET}_{2} N \text{?}\end{matrix}}{\begin{matrix}{( 1 - {FET}_{2} O )}^{*} ( 1 - FETAA ) - \\ {( 1 - {FET}_{2} {O}^{*} FETAA )}^{*} {FET}_{2} NO\end{matrix}} \\ \dot{V} AA = \frac{\begin{matrix}{AAin}^{*} ( 1 - {FET}_{2} NO - {FET}_{2} O - {FET}_{2} {NO}^{*} {FET}_{2} O ) - \\ ( {SGF}^{*} ( 1 + {FET}_{2} CO ) - {N}_{2} Oin - {O}_{2} in - \\ {{FET}_{2} {NO}^{*} {FET}_{2} {O}^{*} ( 1 - {N}_{2} Oin - {O}_{2} in ) )}^{*} FETAA\end{matrix}}{\begin{matrix}{( 1 - {FET}_{2} NO )}^{*} ( 1 - {FET}_{2} O ) - \\ {( 1 - {FET}_{2} {NO}^{*} {FET}_{2} O )}^{*} FETAA\end{matrix}} \\ \text{?} \text{indicates text missing or illegible when filed}$$

Advantages of this method compared to the prior art:

In our method compared to Severinghause (#2)
iv) Patients are maintained with low fresh gas flows (FGF) in a semi-closed circuit, the commonest method of providing anesthesia. No further manipulations by the anesthetist are required.
v) Method uses information normally available in the operating room without additional equipment or monitors.
vi) The calculations can be made with any flow, or combination of flows, of O₂ and N₂O.
vii) Patients can be ventilated or be breathing spontaneously.
viii) Our method can be used to calculate low rates of uptake/absorption such as those of anesthetic vapors Compared to metabolic carts, our method, does not require equipment on addition to that required to anesthetize the patient and there is no need to collect exhaled gas or gas leaving the circuit.

Our method does not require breathing an externally supplied tracer gas. We monitor only routinely available information such as the settings of the O₂ and N₂O flowmeters and the concentrations of gases in expired gas as measured by the standard operating room gas monitor.

Compared to Biro, our approach:
VO₂=O₂in−O₂out (where O₂in and O₂out are
O₂out=TFout*FETO₂TFout=TFin−VO₂
VO₂=O₂in−(TFin−VO₂)*FETO₂

Solving for \dot{V}O₂
VO₂=(O₂in−TFin*FETO₂)/1−FETO₂
where
\dot{V}O₂ is oxygen consumption
TFin is total flow of gas entering the circuit (equivalent to inspiratory flow, VI)
TFout is total flow of gas leaving the circuit (equivalent to expiratory flow, VE)
O₂Out is total flow of O₂ leaving the circuit (equivalent to VO₂out)
O₂in is total flow of O₂ entering the circuit (equivalent to VO₂in)
FETO₂ is the fractional concentration of O₂ in the expired (end-tidal) gas

Our equation takes the same form as that presented by Biro except that Biro's has FIO₂ instead of FETO₂ in analogous places in the numerator and denominator of the term on the right side of the equation. This will clearly result in different values for VO₂ compared to our method. In addition, the difference is that FETO₂ is a steady number during the alveolar phase of exhalation and therefore can be measured and its value is representative of alveolar gas whereas FIO₂ is not a steady number; FIO₂ varies during inspiration and no value at any particular time during inspiration is representative of inspired gas.

Compared to Viale, our method does not require FIO₂, FEN₂, FIN₂ or the patient's gas flows.

Compared to Bengston, our method does not require knowledge of the patient's weight or duration of anesthesia. Our method can be performed with any ratio of O₂/N₂O flow into the circuit. Our method does not require expired gas collection or measurements of gas volume.

Compared to methods by Lowe, Lin or Pestana, our method uses only routinely available information such as the flowmeter settings and end tidal O₂ concentrations. It does not require any invasive procedures.

With these equations, the limiting factor for the precise calculation of gas fluxes is the precision of flowmeters and monitors on anesthetic machines. In addition, leaks, if any, from the circuit and the sampling rate of the gas monitor must be known and taken into account in the calculation. As commercial anesthetic machines are not built to such specifications, we constructed an “anesthetic machine” with precise flowmeters and a lung/circuit model with precisely known flows of O₂ and CO₂ leaving and entering the circuit respectively. We then compared the known fluxes of O₂ and CO₂ with that calculated from the SGF, minute ventilation and the gas concentrations as analyzed by a gas monitor. FIG. 1 shows the Bland-Altman analysis of the results.

We claim:

1) A precise method for determining gas flux calculations and gas pharmacokinetics during low flow anesthesia, one example of which is to institute for dosed circuit anesthesia and for example for a process for determining gas(x) consumption, wherein said gas(x) is selected from;
a) an anesthetic such as but not limited to;
i) N₂O;
ii) sevoflurane;
iii) isoflurane;
iv) halothane;
v) desflurame; or the like
b) Oxygen (O₂);
and further comprising the relationships described in relation to Models I to IV and variations thereof described in the disclosure.

2) A method of determining oxygen consumption, and/or CO₂ production in a subject breathing via a partial rebreathing circuit by the use of information derived from gas flow and composition of gas entering a partial rebreathing circuit and tidal monitor gas concentration readings.

3) A method of determining of oxygen consumption, anesthetic gas absorption and CO₂ production in a subject breathing via a partial rebreathing circuit by the use of information derived from gas flow and composition of gas entering a partial rebreathing circuit and tidal monitor gas concentration readings.

4) The method of claim 2 where the circuit is a circle anesthetic circuit or any anesthetic circuit with CO₂ absorber in the circuit

5) The method of claim 3 where the circuit is a circle anesthetic circuit or any anesthetic circuit with CO₂ absorber in the circuit

6) The process of claim 1 with the use of any of the equations disclosed herein in models 14, including any of the intermediate equations used.

7) Use of any of the following equations or their intermediate equations, for determination of \dot{V}O₂
$$\begin{matrix}\dot{V} {O}_{2} = SGF * ( {FsO}_{2} - {FETO}_{2} ) / ( 1 - {FETO}_{2} ) & ( 4 ) \\ \dot{V} {O}_{2} = \frac{{O}_{2} in - SGF \times {FETO}_{2}}{1 - ( 1 - \frac{SGF}{\dot{V} E} ) {FETO}_{2}} & ( 7 ) \\ \dot{V} {O}_{2} = \frac{( 1 - {FETN}_{2} O ) * {O}_{2} in - ( SGF - {N}_{2} Oin ) * {FETO}_{2}}{1 - ( 1 - \frac{SGF}{\dot{V} E} ) * {FETO}_{2} - {FETN}_{2} O} & ( AA6 ) \\ \dot{V} {O}_{2} = \frac{( I - {FETN}_{2} O - FETAA ) * {O}_{2} in - ( {SGF\_N}_{2} Oin - AAin ) * {FETO}_{2}}{1 - a * {FETO}_{2} - {FETN}_{2} O - FETAA} & ( AA8 ) \\ \dot{V} {O}_{2} = \frac{( 1 - {FETN}_{2} O - FETAA ) * {O}_{2} in - ( SGF - {N}_{2} Oin - AAin ) * {FETO}_{2}}{1 - b * {FETO}_{2} - {FETN}_{2} O - FETAA} & ( AA16 ) \\ VO 2 = \frac{O 2 in - ( SGF + SGF * FETCO 2 ) * {FETO}_{2}}{1 - {FETO}_{2}} & ( 11 ) \\ \dot{V} {O}_{2} = \frac{\begin{matrix}{O}_{2} in * ( 1 - {FETN}_{2} O ) - \\ ( SGF * ( 1 + {FETCO}_{2} ) - {N}_{2} Oin ) * {FETO}_{2}\end{matrix}}{1 - {FETN}_{2} O - {FETO}_{2}} & ( 30 ) \\ \dot{V} O 2 = \frac{\begin{matrix}O 2 in ( 1 - {FETN}_{2} O - {FETAAFETN}_{2} O * FETAA ) \\ ( SGF ( 1 + FETCO ) - {N}_{2} Oin - {AAinFETN}_{2} ) * \\ FETAA ( 1 - {N}_{2} Oin - AAin ) FETO\end{matrix}}{( 1 - {FETN}_{2} O ) * ( 1 - FETAA ) ( 1 - {FETN}_{2} O * FETAA ) {FETO}_{2}} \\ \dot{V} O 2 = \frac{\begin{matrix}{O}_{2} in * \\ ( 1 - {FETN}_{2} O - FETAA - {FETN}_{2} O * FETAA ) - \\ ( SGF * ( 1 + {FETCO}_{2} ) - {N}_{2} Oin - AAin - \\ {FETN}_{2} O * FETAA * ( 1 - {N}_{2} Oin - AAin )\end{matrix}}{\begin{matrix}( 1 - {FETN}_{2} O ) * ( 1 - FETAA ) - \\ ( 1 - {FETN}_{2} O * FETAA ) * {FETO}_{2}\end{matrix}} & ( 11 )\end{matrix}$$

8) Use of any of the following equations or their intermediate equations, for determination of \dot{V}N₂O
$$\begin{matrix}\dot{V} {N}_{2} O = \frac{{N}_{2} Oin - ( SGF - a \dot{V} {O}_{2} ) * {FETN}_{2} O}{1 - {FETN}_{2} O} & ( AA5 ) \\ \dot{V} {N}_{2} O = \frac{\begin{matrix}( 1 - ( 1 - \frac{SGF}{\dot{V} E} ) * {FETO}_{2} ) * {N}_{2} Oin - \\ ( SGF - {O}_{2} in ) * {FETN}_{2} O\end{matrix}}{1 - ( 1 - \frac{SGF}{\dot{V} E} ) * {FETO}_{2} - {FETN}_{2} O} & ( AA7 ) \\ \dot{V} {N}_{2} O = \frac{( 1 - a * {FETO}_{2} - FETAA ) * {N}_{2} Oin - ( SGF - a * {O}_{2} in - AAin ) * {FETN}_{2} O}{1 - a * {FETO}_{2} - {FETN}_{2} O - FETAA} & ( AA9 ) \\ \dot{V} {N}_{2} O = \frac{( 1 - b * {FETO}_{2} ) * {N}_{2} Oin - ( SGF - {O}_{2} in ) * {FETN}_{2} O}{1 - b * {FETO}_{2} - {FETN}_{2} O} \\ Where b = 1 - RQ ( 1 - ( 1 - \frac{SGF}{\dot{V} E} ) ) = 1 - RQ * \frac{SGF}{\dot{V} E} & ( AA15 ) \\ \dot{V} {N}_{2} O = \frac{( 1 - b * {FETO}_{2} - FETAA ) * {N}_{2} Oin - ( SGF - b * {O}_{2} in - AAin ) * {FETN}_{2} O}{1 - b * {FETO}_{2} - {FETN}_{2} O - FETAA} \\ VNO = \frac{\begin{matrix}{N}_{2} Oin * \\ ( 1 - {FETO}_{2} - FETAA - {FETO}_{2} * FETAA ) - \\ ( \begin{matrix}SGF ( 1 + {FETCO}_{2} ) - {O}_{2} in - AAin - \\ {FETO}_{2} * FETAA * ( 1 - {O}_{2} in - AAin )\end{matrix} ) \\ {FETN}_{2} O\end{matrix}}{\begin{matrix}( 1 - {FETO}_{2} ) * ( 1 - FETAA ) - \\ ( 1 - {FETO}_{2} * FETAA ) * {FETN}_{2} O\end{matrix}} & ( AA17 )\end{matrix}$$

9) Use of any of the following equations or their intermediate equations, for determination of \dot{V}AA
$$\dot{V} AA = \frac{( 1 - a * {FETO}_{2} - {FETN}_{2} O ) * AAin - ( SGF - a * {O}_{2} in - {N}_{2} Oin ) * FETAA}{1 - a * {FETO}_{2} - {FETN}_{2} O - FETAA} \\ where a = 1 - \frac{SGF}{\dot{V} E} \\ \dot{V} AA = \frac{( 1 - b * {FETO}_{2} - {FETN}_{2} O ) * AAin - ( SGF - b * {O}_{2} in - {N}_{2} Oin ) * FETAA}{1 - b * {FETO}_{2} - {FETN}_{2} O - FETAA} \\ Where b = 1 - RQ ( 1 - ( 1 - \frac{SGF}{\dot{V} E} ) ) = 1 - RQ * \frac{SGF}{\dot{V} E} \\ \dot{V} AA = \frac{\begin{matrix}AAin * \\ ( 1 - FETNO - FETO - FETNO * FETO ) - \\ ( \begin{matrix}SGF * ( 1 + FETCO ) - {N}_{2} Oin - {O}_{2} in - \\ FETNO * FETO * ( 1 - {N}_{2} Oin - {O}_{2} in )\end{matrix} ) * \\ FETAA\end{matrix}}{\begin{matrix}( 1 - FETNO ) * ( 1 - FETO ) - \\ ( 1 - FETNO * FETO ) * FETAA\end{matrix}} \qquad ( AA10 )$$
